SEQUENCE LISTING (3 sequences)

SEQ ID NO: 1 - DNA, 22 bp, Artificial Sequence
  Description of Artificial Sequencebeta-globin-353F PCR amplification primer
gtgcacctgactcctgaggaga

SEQ ID NO: 2 - DNA, 21 bp, Artificial Sequence
  Description of Artificial Sequencebeta-globin-455R PCR amplification primer
ccttgataccaacctgcccag

SEQ ID NO: 3 - DNA, 26 bp, Artificial Sequence
  Description of Artificial Sequencedual labeled fluorescent PCR probe betaglobin-402T
naggtgaacgtggatgaagttggtgn

(19) United States
(12) Patent Application Publication
(10) Pub. No.: US 2004/0086864 A1
(43) Pub. Date: May 6, 2004

(54) Novel classification methods for pleural effusions

(75) Inventors: Yuk Ming Dennis Lo, Kowloon (HK); Michael Ho-Ming Chan, Tai Po New Territories (HK)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Chinese University of Hong Kong, Shatin (HK)

(21) Appl. No.: 10/278,278

(22) Filed: Oct. 22, 2002

(51) Int. Cl. C12Q 1/68; C12P 19/34

(52) U.S. Cl. 435/6; 435/91.2

(57) ABSTRACT

This invention relates to the detection of nucleic acids in the pleural fluids of a patient suffering from a pleural effusion for the classification of the pleural effusion.

### FIELD OF THE INVENTION

This invention relates to the detection of nucleic acids in the pleural fluids of a patient suffering from a pleural effusion for the classification of the pleural effusion.

### BACKGROUND OF THE INVENTION

Pleural effusions represent a common diagnostic challenge to clinicians. There can be various causes leading to the formation of pleural effusions including congestive heart failure, end-stage renal failure, pulmonary tuberculosis, empyema, chest infection, and malignant neoplasms. Pleural effusions can be classified into exudative and transudative effusions according to their different pathophysiological mechanisms. In a fluid retention or overload state such as congestive heart failure or end-stage renal failure, the excessive intra-vascular fluid will increase the hydrostatic pressure. It will eventually lead to pump failure and congestion of the pulmonary vasculature. This effect, in turn, will cause fluid sequestration into the pleural cavity causing transudative pleural effusion. In the presence of hypoproteinemia, the decrease in plasma colloid osmotic pressure will cause leakage of extra-cellular fluid into the interstitial space leading to the formation of transudative pleural effusion. Therefore, theoretically, transudative pleural effusion shall have a limited inflammatory or cellular element as its pathophysiological mechanism is purely of abnormal fluid and osmotic dynamics. On the other hand, infective and malignant causes such as chest infection, empyema, pulmonary tuberculosis, and lymphangitis carcinomatosis due to infiltration of pulmonary secondary deposits will induce a variable but significant degree of inflammatory and cellular responses into the pleural cavity. The resultant effusions are thus exudative in nature.

Several research groups have attempted to tackle the diagnostic challenge posed by pleural effusions (Saitoh et al., Am. J. of Medicine, 103:400-404 (1997), Yang et al., J. Clin. Oncol 16(2):567-573 (1998), Nagesh et al., Chest, 119(6):1737-1741 (2001), Villegas et al., Chest 118(5):1355-1364 (2000)). For example, Light et al., had proposed the use of four markers, including serum lactate dehydrogenase activity and total protein concentration, as well as the same analytes in pleural fluid to calculate the pleural fluid to serum ratios, in order to formulate the well-known Light's criteria for exudative effusions. These criteria include: pleural fluid to serum total protein ratio greater than 0.5; pleural fluid to serum lactate dehydrogenase ratio greater than 0.6; and pleural fluid lactate dehydrogenase activity greater than 200 IU/L, later modified to be greater than two-thirds of the upper normal reference interval in serum (Light RW et al., Ann Intern Med 1972; 77:507-13) The original study conducted by Light et al three decades ago consisted of 150 patients giving a diagnostic sensitivity of 99% and specificity of 98% for exudative effusions. However, other prospective studies reported much lower diagnostic specificities ranging from 65 to 86 percent (Hirsch A et al., Thorax 1979; 34:106-12; Peterman TA et al, JAMA 1984; 252:1051-3; Roth et al., Chest 1990; 98:546-9). Researchers made attempts to modify the ingredients of the original Light's criteria hoping that there would be an increase in the diagnostic efficacy. All of them had to use multiple markers, although the diagnostic efficacy is only comparable to the original Light's criteria (Romero S et al., Chest 1993; 104:399-404; Vives M et al., Chest 1996; 109:1503-7; Heffner J E et al., Chest 1997; 111:970-80). Thus, for routine practice in many hospitals, clinicians continue to rely on the 30-year-old Light's criteria.

There has been much recent research interest in using plasma cell-free DNA in a quantitative way for prenatal diagnosis, cancer testing, acute trauma, and monitoring of transplantation (Lo Y M D et al., N Engl J Med 1998; 339:1734-8; Lo Y M D et al., Cancer Res 1999; 59:1188-91; Lo Y M D et al., Clin Chem 2000; 46:319-23; Lo Y M D et al., Lancet 1998; 351: 1329-30; Lui Y Y N et al., Clin Chem 2002; 48:421-27). Investigation on cell-free DNA has also been carried out in biological fluids other than plasma, for example, urine (Botezatu I et al., Clin Chem 2000; 46:1078-84). In this application, for the first time, it is demonstrated that the detection and quantification of nucleic acids in pleural fluid can be used for the classification of pleural effusions as transudative or exudative.

### BRIEF SUMMARY OF THE INVENTION

In one aspect of the invention, a method of classifying a pleural effusion in a subject as transudative or exudative is disclosed. The method comprises obtaining a sample of pleural fluids from a patient suffering from a pleural effusion, detecting the concentration of human nucleic acid in the sample, with the proviso that the nucleic acid is not overexpressed in cancer cells and is not telomerase or adenosine deaminase nucleic acid, and classifying the pleural effusion as transudative or exudative by comparing the concentration of nucleic acid in the sample to a standard. In one embodiment, the exudative effusion is further classified as a malignant effusion or an infective effusion.

In another aspect of the invention, the patient who has a pleural effusion is suffering from a disease selected from the group consisting of congestive heart failure, end-stage renal failure, pulmonary tuberculosis, empyema, chest infection, malignant neoplasm, pulmonary embolism, pneumonia, liver disease, kidney disease, and lymphangitis carcinomatosis

In another aspect of the invention, the method of classifying a pleural effusion in a subject comprises obtaining a sample and detecting the concentration of nucleic acid in the sample. In one embodiment, the nucleic acid in the sample is DNA. In another embodiment, the nucleic acid in the sample is RNA. In another embodiment, the DNA detected is the β-globin gene DNA.

In another aspect of the invention, the method of classifying a pleural effusion in a subject further comprises the step of amplifying the nucleic acid in the sample. In one embodiment, the nucleic acid to be amplified is DNA and the DNA is amplified using PCR. In another embodiment, the DNA is amplified using real-time PCR. In another embodiment, the nucleic acid to be amplified is RNA and the RNA is amplified using reverse transcriptase PCR. In another embodiment, the RNA is amplified using reverse transcriptase real-time PCR.

Definitions

A “pleural effusion” refers to a condition characterized by an excess quantity of fluid in the pleural space. The pleural space lies between the lung and the chest wall. A pleural effusion can be classified as transudative or exudative. Exudative pleural effusions can further be classified as malignant or infective.

A “transudative pleural effusion” refers to an effusion that is caused by the alteration of systemic factors that influence the formation and absorption of pleural fluid. A resulting imbalance between the venous-arterial pressure and the pressure within the pleural space cause excess fluid to accumulate in the pleural space. Causes of transudative effusions include, but are not limited to, cardiac failure, e.g., left ventricular failure, pulmonary embolism, liver disease, e.g., cirrhosis, kidney disease, e.g., nephrotic syndrome, and lymphatic blockade produced by cancer.

An exudative pleural effusion refers to an effusion that is caused by the alteration of local factors that influence the formation and absorption of pleural fluid. Inflammation, infection and cancer are causal factors for exudative pleural effusions. Bacterial pneumonia, viral infection, malignancy, and pulmonary embolism are the leading causes of exudative effusions. A “malignant pleural effusion” refers to an exudative effusion caused by, for example, cancers, such as carcinomas of the breast, lung, gastrointestinal tract or ovary and by lymphomas. An “infective pleural effusion” refers to an effusion caused by infections, such as tuberculosis.

The “predictive cut-off concentration” is the concentration of nucleic acid in the pleural fluid that can be used to classify a pleural effusion as transudative or exudative or an exudative effusion as infective or malignant. For example, if DNA concentration levels are above the predictive cut-off concentration for discriminating between transudative and exudative effusions, an effusion can be classified as exudative. Alternatively, if DNA concentrations are below the predictive cut-off concentration for discriminating between transudative and exudative effusions, an effusion is then classified as transudative. The “predictive cut-off concentration” can thereby act as the control.

The phrase “a sample of pleural fluid”, as used herein, refers to a pleural fluid sample obtained from a subject. Frequently the sample will be a “clinical sample” which is a sample derived from a subject with a pleural effusion or suspected of having a pleural effusion (a “patient”).

“Nucleic acid” refers to a deoxyribonucleotide or ribonucleotide polymer in either single- or double-stranded form, and unless otherwise limited, would encompass known analogs of natural nucleotides that can function in a similar manner as naturally occurring nucleotides.

The nucleic acids may be single stranded or double stranded, as specified, or contain portions of both double stranded or single stranded sequence. The depiction of a single strand also defines the sequence of the complementary strand; thus the sequences described herein also provide the complement of the sequence. The nucleic acid may be DNA, both genomic and cDNA, RNA or a hybrid, where the nucleic acid may contain combinations of deoxyribo- and ribo-nucleotides, and combinations of bases, including uracil, adenine, thymine, cytosine, guanine, inosine, xanthine, hypoxanthine, isocytosine, isoguanine, etc. “Transcript” typically refers to a naturally occurring RNA, e.g., a pre-mRNA, hnRNA, or mRNA. As used herein, the term “nucleoside” includes nucleotides and nucleoside and nucleotide analogs, and modified nucleosides such as amino modified nucleosides. In addition, “nucleoside” includes non-naturally occurring analog structures. Thus, e.g. the individual units of a peptide nucleic acid, each containing a base, are referred to herein as a nucleoside.

The terms “hybridize(s) specifically” or “specifically hybridize(s)” refer to the binding, duplexing, or hybridizing of a molecule to a particular nucleotide sequence under stringent hybridization conditions when that sequence is present in a complex mixture (e.g., total cellular or library DNA or RNA). The terms also refer to complementary hybridization between an oligonucleotide (e.g., a primer or labeled probe) and a target sequence. The terms specifically embrace minor mismatches that can be accommodated by reducing the stringency of the hybridization conditions to achieve the desired priming for the PCR polymerases or detection of hybridization signal.

The term “substantially identical” indicates that two or more nucleotide sequences share a majority of their sequences. Generally, this will be at least about 90% of their sequences and preferably about 95% of their sequences. Another indication that the sequences are substantially identical is if they hybridize to the same nucleotide sequence under stringent conditions (see, e.g., Sambrook and Russell, eds, Molecular Cloning: A Laboratory Manual, 3rd Ed, vols. 1-3, Cold Spring Harbor Laboratory Press, 2001; and Current Protocols in Molecular Biology, Ausubel, ed. John Wiley & Sons, Inc. New York, 1997). Stringent conditions are sequence-dependent and will be different in different circumstances. Generally, stringent conditions are selected to be about 5° C. (or less) lower than the thermal melting point (Tₘ) for the specific sequence at a defined ionic strength and pH. The Tₘ of a DNA duplex is defined as the temperature at which 50% of the nucleotides are paired and corresponds to the midpoint of the spectroscopic hyperchromic absorbance shift during DNA melting. The Tₘ indicates the transition from double helical to random coil

The term “oligonucleotide” refers to a molecule comprised of two or more deoxyribonucleotides or ribonucleotides, such as primers, probes, and other nucleic acid fragments. The exact size of an oligonucleotide depends on many factors and the ultimate function or use of the oligonucleotide. “Adding” an oligonucleotide refers to joining an oligonucleotide to another nucleic acid molecule. Typically, adding the oligonucleotide is performed by ligating the oligonucleotide using a DNA ligase.

The term “primer” refers to an oligonucleotide, whether natural or synthetic, capable of acting as a point of initiation of DNA synthesis under conditions in which synthesis of a primer extension product complementary to a nucleic acid strand is induced, i.e., in the presence of four different nucleoside triphosphates and an agent for polymerization (such as DNA polymerase or reverse transcriptase) in an appropriate buffer and at a suitable temperature. A primer is preferably a single-stranded oligodeoxyribonucleotide sequence. The appropriate length of a primer depends on the intended use of the primer but typically ranges from about 15 to about 30 nucleotides. Short primer molecules generally require cooler temperatures to form sufficiently stable hybrid complexes with the template. A primer need not reflect the exact sequence of the template but must be sufficiently complementary to specifically hybridize with a template.

“Probe” refers to an oligonucleotide which binds through complementary base pairing to a subsequence of a target nucleic acid. It will be understood by those skilled in the art that probes will typically substantially bind target sequences lacking complete complementarity with the probe sequence depending upon the stringency of the hybridization conditions. The probes are typically directly labeled (e.g., with isotopes or fluorescent moieties) or indirectly labeled such as with digoxigenin or biotin. By assaying for the presence or absence of the probe, one can detect the presence or absence of the target.

### BRIEF DESCRIPTION OF THE DRAWINGS

FIG. 1. Receiver Operator Characteristic curve analysis of pleural fluid DNA concentrations for differentiating between the exudative and transudative groups. Values indicated on the x- and y-axes are expressed in percentages. The area under the curve is 0.963 (CI:0.851-0.995).

FIG. 2. Receiver Operator Characteristic curve analysis of pleural fluid DNA concentrations for differentiating between infective and malignant effusions. Values indicated on the x- and y-axes are expressed in percentages. The area under the curve is 0.726 (CI:0.53-0.874).

FIG. 3. Pleural fluid β-globin gene DNA concentrations in subjects with malignant, infective, or transudative pleural fluid concentrations. Pleural fluid DNA concentrations as determined by real-time quantitative PCR for the β-globin gene (y-axis) are plotted against pleural effusion categories (x-axis). The lines inside the boxes denote medians whilst the boxes mark the interval between the 25^{th} and 75^{th} percentiles. The whiskers denote the interval between the 10^{th} and 90^{th} percentiles.

FIG. 4. Correlation between pleural fluid β-globin gene DNA concentration and pleural fluid lactate dehydrogenase activity. Pleural fluid DNA concentrations as determined by real-time quantitative PCR for the β-globin gene (y-axis) are plotted against pleural fluid lactate dehydrogenase activity (x-axis).

FIG. 5. Correlation between pleural fluid β-globin gene DNA concentration and pleural fluid total protein concentrations. Pleural fluid DNA concentrations as determined by real-time quantitative PCR for the β-globin gene (y-axis) are plotted against pleural fluid total protein concentration (x-axis).

FIG. 6. Mode of Median of pleural fluid β-globin gene DNA concentrations in subjects with malignant, infective, or transudative pleural fluid concentrations. The multiples of median of pleural fluid DNA concentrations as determined by real-time quantitative PCR for the β-globin gene (y-axis) are plotted against pleural effusion categories (x-axis). The lines inside the boxes denote medians whilst the boxes mark the interval between the 25^{th} and 75^{th} percentiles. The whiskers denote the interval between the 10^{th} and 90^{th} percentiles.

FIG. 7. Receiver Operator Characteristic curve for multiples of median (MOM) of pleural fluid DNA concentrations for differentiating between the exudative and transudative groups. Values indicated on the x- and y-axes are expressed in percentages. The area under the curve is 0.963 (CI:0.851-0.995).

### DETAILED DESCRIPTION OF THE INVENTION

This invention pertains to the surprising discovery that levels of nucleic acid present in the pleural fluids can be used to classify a pleural effusion as transudative or exudative. An exudative effusion can then be further classified as malignant or infective. Without being bound by theory, it is believed that the possible origins of pleural fluid DNA could be due to ultra-filtration from the plasma or a local production from dying or apoptotic cells.

Using the methods of the present invention, nucleic acid present in the pleural fluid of a subject is quantified and a determination is made whether the effusion is exudative, e.g., malignant or infective, or transudative in nature. Accordingly, the present invention provides a method of distinguishing between a transudative and exudative pleural effusion and/or a malignant and infective pleural effusion based on nucleic acid concentration in a sample of pleural fluid. For example, a low concentration of nucleic acid in the pleural fluids indicates that the patient is suffering from a transudative effusion.

The present invention also provides a method of diagnosing an exudative pleural effusion in a patient. For example, a high concentration of nucleic acid in the pleural fluids, e.g., a concentration above the predictive cut-off concentration, indicates that a patient is suffering from an exudative effusion. Alternatively, the present invention also provides a method of diagnosing a transudative pleural effusion in a patient. For example, a low concentration of nucleic acid in the pleural fluids, e.g., a concentration below the predictive cut-off concentration, indicates that the patient is suffering from a transudative effusion.

Selecting a Patient Population

The present invention provides methods for classifying a pleural effusion as transudative or exudative in nature in a patient suffering from a pleural effusion. The present invention further provides methods for classifying an exudative pleural effusion as malignant or infective in a patient suffering from a pleural effusion.

A skilled practitioner will know how to determine whether a patient is suffering from a pleural effusion. Typically, the abnormal accumulation of fluid in the pleural space is associated with an accompanying disease in a subject. Accordingly, a practitioner might suspect a pleural effusion in a subject based on his or her medical history. The subject may also have symptoms associated with pleural effusions. Symptoms include shortness of breath, a sharp chest pain which worsens with coughing or deep breaths, cough, hiccups, rapid breathing, and abdominal pain.

A diagnosis of pleural effusion can be confirmed by tests that are well known in the art. These include chest X-rays, thoracic CTs, Chest MRIs, pleural biopsies, diagnostic thoracocentesis, percussion, and ultrasound of the chest. For example, abnormal accumulation of pleural fluid can be located in a subject by percussion. A subject sits at a table, leaning against it with his or her arms resting on the tabletop. A practitioner then places one finger on the subject's back and taps against this finger with a finger from the other hand. If the lungs are filled with fluid, a dull sound will be emitted. If the lungs are filled with air, the sound will be hollow.

Obtaining Pleural Fluid Samples and DNA Extraction

Pleural fluid samples are obtained from the patients described in the present invention. Pleural fluid samples can be obtained by methods known in the art, such as thoracocentesis.

In thoracocentesis, a needled catheter is introduced into the pleural space through an incision in the chest cavity and fluid is positively drawn out through the catheter using a syringe or a vacuum source. In some embodiments, a second syringe may be used. Once pleural fluid aspirates into the first needle, a larger needle is inserted to drain the fluid more efficiently. Other approaches to removing fluid from the pleural space include surgically implanting a chest tube or using a special catheter device that can be implanted in the pleural space for extended periods of time (see U.S. Pat. No. 5,484,401).

After collection, the pleural fluids are processed according to standard procedure. For example, in some methods, the pleural fluids are collected into polypropylene tubes and fractionated by centrifugation. The samples are then stored at −20° C. until further use. After collection, DNA is extracted from the pleural fluids according to standard methods. For example, DNA can be extracted using QIAamp Blood kit (Qiagen) following the blood and body fluid protocol according to the manufacturer's recommendation. Typically, about 600 to 800 μL pleural fluid is used for DNA extraction.

Nucleic Acid Detection Methods

The nucleic acids detected in the methods of the invention are typically from about 40 nucleotides in length to several thousand nucleotides in length. Usually, the nucleic acids are from about 80 to about 200 nucleotides.

After nucleic acid, e.g., DNA or RNA, has been isolated from pleural fluids, any of the conventional DNA or RNA detection methods can be used for the detection and quantification, e.g., amount or concentration, of nucleic acid. In a preferred embodiment, any means for detecting low copy number nucleic acids are used to detect the nucleic acids of the present invention. Means for detecting and quantifying low copy number nucleic acids include analytic biochemical methods such as electrophoresis, capillary electrophoresis, high performance liquid chromatography (HPLC), thin layer chromatography (TLC), hyperdiffusion chromatography, mass spectroscopy and the like. These methods are well known in the art and are thus not described in detail (See for example, U.S. Pat. Nos. 6,013,422, 6,261,781, 6,268,146, or 5,885,775).

The methods of the present invention typically but not always rely on amplification or signal amplification methods for the detection of the nucleic acids. One of skill will recognize that amplification of target sequences in a sample may be accomplished by any known method, such as ligase chain reaction (LCR), Qβ-replicase amplification, transcription amplification, and self-sustained sequence replication, each of which provides sufficient amplification.

In one embodiment of the present invention, PCR is used to detect nucleic acids circulating in the pleural fluids. Typically, a greater concentration of nucleic acid species will be present in pleural fluid resulting from an exudative pleural effusion than the concentration of nucleic acid species in pleural fluid resulting from a transudative pleural effusion. One of skill will know how to use standard methods to prepare primers for amplification of a known nucleic acid sequence and to subsequently amplify the sequence and visualize the products on a gel. The PCR process is well known in the art. For a review of PCR methods and protocols, see, e.g., Innis, et al. eds. PCR Protocols. A Guide to Methods and Application (Academic Press, Inc., San Diego, Calif. 1990). PCR reagents and protocols are also available from commercial vendors, such as Roche Molecular Systems. The nucleic acids detected can be DNA or RNA molecules. In particular embodiments of the invention, RNA molecules are detected. The detected RNA molecules can also be RNA transcribed from genomic sequences, but which do not encode functional polypeptides. The first step in the amplification is the synthesis of a DNA copy (cDNA) of the region to be amplified. Reverse transcription can be carried out as a separate step, or in a homogeneous reverse transcription-polymerase chain reaction (RT-PCR), a modification of the polymerase chain reaction for amplifying RNA. Methods suitable for PCR amplification of ribonucleic acids are described by Romero and Rotbart in Diagnostic Molecular Biology: Principles and Applications pp.401-406, Persing et al., eds., (Mayo Foundation, Rochester, Minn. 1993); Rotbart et al,. U.S. Pat. No. 5,075,212 and Egger et al., J. Clin. Microbiol. 33:1442-1447 (1995).

The primers used in the methods of the invention are preferably at least about 15 nucleotides to about 50 nucleotides in length, more preferably from about 15 nucleotides to about 30 nucleotides in length.

To amplify a target nucleic acid sequence in a sample by PCR, the sequence must be accessible to the components of the amplification system. In general, this accessibility is ensured by isolating the nucleic acids from the sample. A variety of techniques for extracting nucleic acids, from biological samples are known in the art and described above.

The first step of each cycle of the PCR involves the separation of the nucleic acid duplex formed by the primer extension. Once the strands are separated, the next step in PCR involves hybridizing the separated strands with primers that flank the target sequence. The primers are then extended to form complementary copies of the target strands. For successful PCR amplification, the primers are designed so that the position at which each primer hybridizes along a duplex sequence is such that an extension product synthesized from one primer, when separated from the template (complement), serves as a template for the extension of the other primer. The cycle of denaturation, hybridization, and extension is repeated as many times as necessary to obtain the desired amount of amplified nucleic acid (amplicon).

In the preferred embodiment of the PCR process, strand separation is achieved by heating the reaction to a sufficiently high temperature (˜95° C.) for a sufficient time to cause the denaturation of the duplex but not to cause an irreversible denaturation of the polymerase (see U.S. Pat. No. 4,965,188). Template-dependent extension of primers in PCR is catalyzed by a polymerizing agent in the presence of adequate amounts of four deoxyribonucleoside triphosphates (typically dATP, dGTP, dCTP, and dTTP) in a reaction medium comprised of the appropriate salts, metal cations, and pH buffering system. Suitable polymerizing agents are enzymes known to catalyze template-dependent DNA synthesis. In the present invention, the initial template for primer extension is typically first strand cDNA that has been transcribed from RNA. Reverse transcriptases (RTs) suitable for synthesizing a cDNA from the RNA template are well known.

PCR is most usually carried out as an automated process with a thermostable enzyme. In this process, the temperature of the reaction mixture is cycled through a denaturing region, a primer annealing region, and an extension reaction region automatically.

The nucleic acids of the invention can also be detected using other standard techniques, well known to those of skill in the art. Although the detection step is typically preceded by an amplification step, amplification is not required in the methods of the invention. For instance, the nucleic acids can be identified by size fractionation (e.g., gel electrophoresis). Alternatively, the target nucleic acids can be identified by sequencing according to well known techniques. Alternatively, oligonucleotide probes specific to the target nucleic acids can be used to detect the presence of specific fragments.

Sequence-specific probe hybridization is a well known method of detecting desired nucleic acids in a sample comprising cells, biological fluids and the like. Under sufficiently stringent hybridization conditions, the probes hybridize specifically only to substantially complementary sequences. The stringency of the hybridization conditions can be relaxed to tolerate varying amounts of sequence mismatch. If the target is first amplified, detection of the amplified product utilizes this sequence specific hybridization to insure detection of only the correct amplified target, thereby decreasing the chance of a false positive.

A number of hybridization formats are well known in the art, including but not limited to, solution phase, solid phase, oligonucleotide array formats, mixed phase, or in situ hybridization assays. In solution (or liquid) phase hybridizations, both the target nucleic acid and the probe or primers are free to interact in the reaction mixture. Techniques such as real-time PCR systems have also been developed that permit analysis, e.g., quantification, of amplified products during a PCR reaction. In this type of reaction, hybridization with a specific oligonucleotide probe occurs during the amplification program to identify the presence of a target nucleic acid. Hybridization of oligonucleotide probes ensure the highest specificity due to thermodynamically controlled two state transition. Examples for this assay formats are fluorescence resonance energy transfer hybridization probes, molecular beacons, molecular scorpions, and exonuclease hybridization probes (reviewed in Bustin S M. J. Mol. Endocrin. 25:169-93 (2000)).

In solid phase hybridization assays, either the target or probes are linked to a solid support where they are available for hybridization with complementary nucleic acids in solution. Exemplary solid phase fornats include Southern or Northern hybridizations, dot blots, arrays, chips, and the like. In situ techniques are particularly useful for detecting target nucleic acids in chromosomal material (e.g., in metaphase or interphase cells). The following articles provide an overview of the various hybridization assay formats: Singer et al., Biotechniques 4:230 (1986); Haase et al., METHODS IN VIROLOGY, Vol. VII, pp. 189 226 (1984); Wilkinson, IN SITU HYBRIDIZATION, D. G. Wilkinson ed., IRL Press, Oxford University Press, Oxford; and NUCLEIC ACID HYBRIDIZATION: A PRACTICAL APPROACH, Hames, B. D. and Higgins, S. J., eds., IRL Press (1987).

The hybridization complexes are detected according to well known techniques and are not a critical aspect of the present invention. Nucleic acid probes capable of specifically hybridizing to a target can be labeled by any one of several methods typically used to detect the presence of hybridized nucleic acids. One common method of detection is the use of autoradiography using probes labeled with 3H, 125I, 35S, 14C, or 32P, or the like. The choice of radioactive isotope depends on research preferences due to ease of synthesis, stability, and half-lives of the selected isotopes. Other labels include compounds (e.g., biotin and digoxigenin), which bind to anti-ligands or antibodies labeled with fluorophores, chemiluminescent agents, and enzymes. Alternatively, probes can be conjugated directly with labels such as fluorophores, chemiluminescent agents or enzymes. The choice of label depends on sensitivity required, ease of conjugation with the probe, stability requirements, and available instrumentation.

The probes and primers of the invention can be synthesized and labeled using well-known techniques. Oligonucleotides for use as probes and primers may be chemically synthesized according to the solid phase phosphoramidite triester method first described by Beaucage, S. L. and Caruthers, M. H., 1981, Tetrahedron Letts., 22(20):1859 1862 using an automated synthesizer, as described in Needham VanDevanter, D. R., et al. 1984, Nucleic Acids Res., 12:6159 6168. Purification of oligonucleotides can be performed, e.g., by either native acrylamide gel electrophoresis or by anion exchange HPLC as described in Pearson, J. D. and Regnier, F. E., 1983, J. Chrom., 255:137 149.

Detection of the nucleic acid sequences can also be accomplished by means of signal amplification techniques. For example, the branched DNA assay uses a specific probe to a target sequence to identify the presence of the target. The signal is amplified by means of modifications made to the probe which allow many fluorescent detector DNA molecules to hybridize to a target nucleic acid (Chiron Diagnostics).

Any nucleic acid species present in the pleural fluid of a subject can be detected by the methods of the present invention and used as to classify a pleural effusion as transudative or exudative. The exudative pleural effusions can be further classified as malignant or infective. Typically, there will be significant differences in nucleic acid concentration between malignant and transudative effusions, malignant and infective effusions, and infective and transudative effusions. Nucleic acid concentrations will be greatest in infective effusions and smallest in transudative effusions.

Examples of nucleic acids species that can be used in the methods of the present invention include, but are not limited to, the human leukocyte antigen (HLA) locus, Y chromosomal genes (Lee T H et al., Transfusion 2001;41:276-282), blood group antigen genes like RHD (Lo Y M D et al., N. Engl. J. Med. 1998;339:1734-1738), and mitochondrial DNA (Zhong S et al., J. Clin. Pathol. 2000;53:466-469) and mRNA (Poon L L M et al., Clin. Chem. 2000;46:1832-1834; Chen X Q et al., Clin. Cancer Res. 6:3823-3826). Another exemplary marker used in the methods of the present invention is the DNA encoding the β-globin gene. Probes and primers for the detection of the β-globin gene can be synthesized using well-known techniques and are well known in the art, (see Example 2). Probes and primers for the detection of other known nucleic acid species in the pleural fluid of a subject suffering from a pleural effusion can be synthesized using well known techniques (see Example 5).

Methods of Classifying the Pleural Effusion

Once the nucleic acid in the sample has been detected and quantified, the concentration of nucleic acid in the sample is compared to a control. A skilled practitioner can use the comparison to determine if a subject is suffering from transudative or exudative pleural effusion. The greater the concentration of nucleic acid in the sample, the more likely that the effusion is an infective exudative effusion. The smaller the concentration of nucleic acid in the sample, the more likely the effusion is transudative. If the nucleic acid concentration is in a medium range, the more likely the effusion is a malignant exudative effusion.

In order to determine a predictive cut-off concentration that will enable the skilled practitioner to differentiate between the pleural effusion types, the well-known Receiver-Operator-Characteristics (ROC) curve method was used. ROC curve is a plot of sensitivity, wherein the sensitivity refers to the percentage of positive test result in a cohort of subjects with the disease, in y-axis against 100% - specificity, wherein the specificity refers to the percentage negative test result in a cohort of subjects without the disease, in x-axis. The maximum area under the ROC curve (AUC) is unity. Thus, in ROC curve analysis of a test method, the higher the AUC, the greater is the efficiency of the concerned test method to differentiate between disease (in case of exudative effusion) and non-disease (in case of transudative effusion). Similarly, ROC curve analysis can be applied to differentiate between infective effusion and malignant effusion.

Automatic calculation of the sensitivity, specificity, positive predictive value (PPV), negative predictive value (NPV), positive likelihood ratio (LR+), and negative likelihood ratio (LR−), and ROC curve is available in many state of the art statistical programs such as MedCalc or SPSS for Windows, or any other suitable statistical programs. Furthermore, the best cut-off concentration can be chosen from the ROC curve by picking the concentration at which both the sensitivity and specificity are maximized. The greater the LR+, the greater the predictability of a test method to diagnose the disease (in case of exudative effusion). The smaller the LR−, the greater the predictability of a test method to exclude the non-disease (in case of transudative effusion). Similarly, another cut-off pleural fluid DNA concentration can be determined with the respective LR+ and LR− calculated to predict the presence of infective effusion against malignant effusion or vice versa.

Once the best cut-off concentrations are determined, the test method can be applied clinically to classify pleural effusions. The greater the concentration of nucleic acid in the sample, the more likely that the effusion is an infective exudative effusion. The smaller the concentration of nucleic acid in the sample, the more likely the effusion is transudative. If the nucleic acid concentration is in a medium range, the more likely the effusion is a malignant exudative effusion. This exemplifies how pleural fluid DNA can be applied clinically to classify pleural effusions.

### EXAMPLES

The following examples are offered to illustrate, but not to limit the claimed invention.

### Example 1

Study Design and Patients

Patients that presented to the Department of Medicine & Therapeutics and the Department of Clinical Oncology, Prince of Wales Hospital, Hong Kong, with pleural effusions requiring therapeutic or diagnostic aspiration to alleviate or investigate the etiology of the effusions were recruited after obtaining infonned consent.

Twenty mL of pleural fluid and 4 mL of clotted blood were collected from the same setting from each patient at the time of therapeutic tapping. The pleural fluid and clotted blood samples were centrifuged at 1,600 g (Megafuge 1.0R, Heraeus Instruments, Hanau, Germany) for 10 minutes. An aliquot of the supernatants from the pleural fluid and clotted blood samples were used to measure pleural fluid and serum lactate dehydrogenase activity and total protein concentration respectively to calculate the modified Light's criteria. The remaining supernatants from the pleural fluid samples were transferred into polypropylene tubes and were further subjected to micro-centrifugation for 10 minutes at 13,000 g (Eppendorf Centrifuge 5415D, Hamburg, Germany). These re-centrifuged pleural fluid samples were then used for DNA extraction followed by PCR analysis. All samples were processed within 2 hours of sample collection and transferred into polypropylene tubes and stored at −203° C. until further use.

Extraction of the Pleural Fluid DNA

DNA extraction from the above-processed pleural fluid aliquots was performed using a QIAamp Blood Kit (Qiagen, Hilden, Germany) by use of the blood and body fluid protocol according to the manufacturer's recommendations. The volume of pleural fluid used for DNA extraction was 600 to 800 μL per column.

### Example 2

Amplification of the Extracted Pleural Fluid DNA

All of the pleural fluid aliquots were subjected to real-time quantitative PCR amplification for the β-globin gene as described previously (Lo Y M D et al., Am J Hum Genet 1998; 62:768-75). The beta-globin PCR system consists of the amplification primers: SEQ ID NO:1 - beta-globin-354F; 5′-GTG CAC CTG ACT CCT GAG GAG A-3′; SEQ ID NO:2 - beta-globin-455R; 5′-CCT TGA TAC CAA CCT GCC CAG-3′; SEQ ID NO:3 - Dual labeled fluorescent PCR probe beta globin-402T; 5′-(VIC)AAG GTG AAC GTG GAT GAA GTT GGT GG(TAMRA)-3′ (Lo Y M D et al., Am J Hum Genet 1998; 62:768-75). The PCR probe contained a 3′-blocking phosphate group to prevent probe extension during PCR. The volume of extracted pleural fluid DNA used for amplification was 5 μL. Real-time quantitative PCR was performed by use of an Applied Biosystems 7700 Sequence Detector (Applied Biosystems, Foster City, Calif., USA). The theoretical and practical aspects of real-time quantitative PCR have been described in detail elsewhere (Heid C A et al., Genome Res 1996; 6:986-94). Duplicate analyses were performed for each sample, and the mean result was used for further analysis. A calibration curve was analyzed in parallel with each assay. Double-distilled water was used as the negative control for quantitative real-time PCR. The results were expressed as genome-equivalents by use of the conversion factor of 6.6 pg of DNA per cell (Lo Y M D et al., Am J Hum Genet 1999; 64:218-24). Amplification data were analyzed and stored by the Sequence Detection System Software Ver. 1.6.3 (Applied Biosystems, Foster City, Calif., USA). The pleural fluid DNA concentrations expressed in genome-equivalents per milliliter were calculated as described previously (Lo Y M D et al., Am J Hum Genet 1998; 62:768-75).

### Example 3

Data Analysis of Pleural Fluid DNA Concentrations

Data analysis for Spearman correlation, linear regression, and non-parametric Kruskal-Wallis test statistics were performed by the use of SPSS 10.0 for Windows (SPSS). Receiver-Operator Characteristic (ROC) curve was plotted using MedCalc 6.16 statistics program (MedCalc) to determine the best cut-off concentration for pleural fluid DNA. With the cut-off concentration determined, sensitivity, specificity, positive predictive value (PPV), negative predictive value (NPV), positive likelihood ratio (LR+), and negative likelihood ratio (LR−) for pleural fluid DNA and the modified Light's criteria can be calculated using the discharge, microbiological or histological diagnoses as the gold standard. The well-known Light's criteria include: pleural fluid to serum total protein ratio greater than 0.5; pleural fluid to serum lactate dehydrogenase ratio greater than 0.6; and pleural fluid lactate dehydrogenase activity greater than 200 IU/L, later modified to be greater than two-thirds of the upper normal reference interval in serum (Light R W et al., Ann Intern Med 1972; 77:507-13).

### Example 4

Outcome

A total of 41 patients were recruited, of whom 29 patients were from the Department of Medicine & Therapeutics while 12 were from the Department of Clinical Oncology. There were 25 males and 16 females with an age range from 21 to 99 (median=69). The patient demographics with their respective discharge, microbiological or histological diagnoses are presented in Table 1.

TABLE 1

| | Malignant Effusions (19) | Infective Effusions (10) | Transudative Effusions (12) |
|---|---|---|---|
| Male:Female | 13:6 | 7:3 | 5:7 |
| Age Range (median) | 45-86 (69) | 21-93 (47) | 47-99 (69) |
| Diagnosis (number) | Carcinoma of lung (10) | Pulmonary tuberculosis (8) | Congestive heart failure (4) |
| | Non-small cell type (7) | Empyema (1) | End-stage renal failure (8) |
| | Small cell type (2) | Pneumonia (1) | |
| | Adenocarcinoma type (1) | | |
| | Carcinoma of breast (1) | | |
| | Carcinoma of colon (1) | | |
| | Hepatocellular carcinoma (1) | | |
| | Nasopharyngeal carcinoma | | |
| | (1) | | |
| | T-cell lymphoma (1) | | |
| | Unknown primary cancer (4) | | |

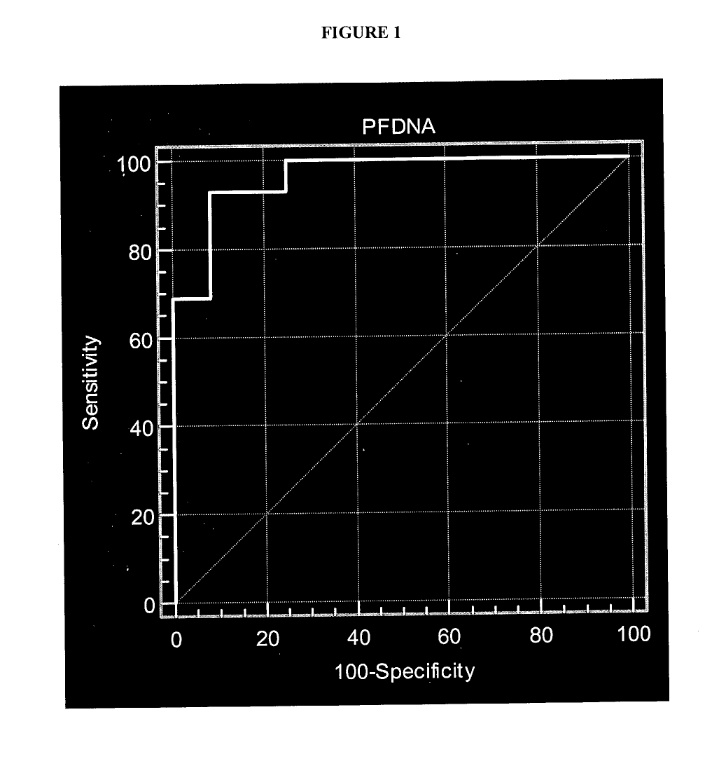

In an exemplary embodiment of the present invention, the ROC curve for pleural fluid DNA concentration to classify between exudative and transudative effusions was plotted and shown in FIG. 1. The area under the curve (AUC) is 0.963 [95% Confidence Interval (95% CI): 0.851-0.995]. The best cut-off concentration for pleural fluid DNA was chosen to be 508.5 genome-equivalents/mL. Pleural fluids with their respective DNA concentrations equal to or above 508.5 genome-equivalents/mL are regarded as exudative effusions while pleural fluids with their respective DNA concentrations below this cut-off are regarded as transudative effusions.

Using this cut-off concentration, 38 out of 41 [sensitivity=93.1% (95% CI: 77.2% -99.0%); specificity=91.7% (95% CI: 61.5% -98.6%)] pleural effusions were correctly classified into exudative and transudative groups when compared to the gold standard. The positive likelihood ratio (LR+) and negative likelihood ratio are 11.17 and 0.08 at this cut-off concentration. Using the modified Light's criteria, 36 out of 41 [sensitivity=96.6%; specificity=66.6%] pleural effusions were correctly classified into exudative and transudative groups when compared to the gold standard. The positive predictive values for pleural fluid DNA and modified Light's criteria are 96.4% and 87.5%, respectively. The negative predictive values for pleural fluid DNA and modified Light's criteria are 84.6% and 88.8%, respectively.

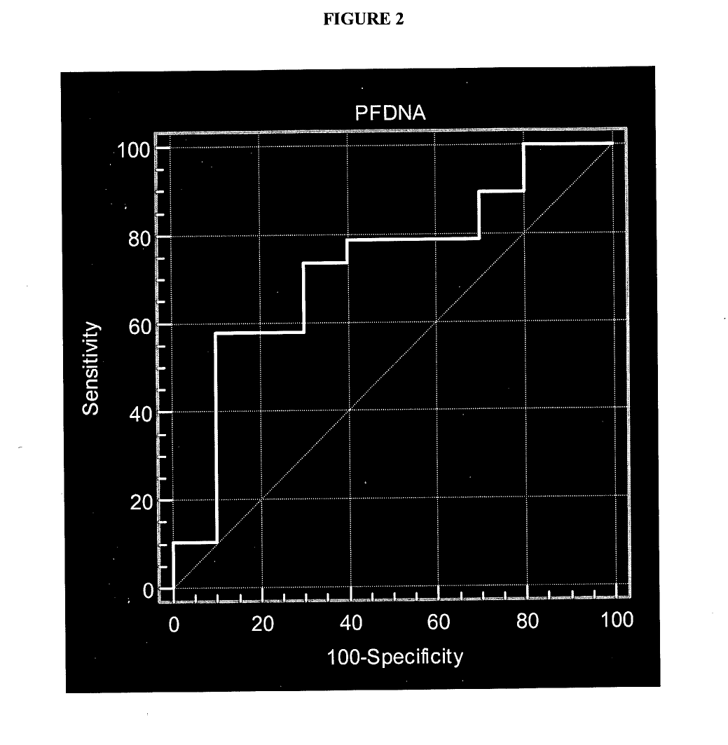

In an exemplary embodiment of the present invention, the ROC curve for pleural fluid DNA concentration to classify between infective and malignant effusions was plotted and shown in FIG. 2. The AUC is 0.726 [95% CI: 0.530-0.874]. The best cut-off concentration for pleural fluid DNA was chosen to be 4221 genome-equivalents/mL. Pleural fluids with their respective DNA concentrations equal to or above 4221 genome-equivalents/mL are more likely to be infective effusions while pleural fluids with their respective DNA concentrations below this cut-off are more likely to be malignant effusions.

Using this cut-off concentration, 11 out of 19 [sensitivity=57.9% (95% CI: 33.5% -79.7%); specificity=90.0% (95% CI: 55.5% -98.3%)] malignant pleural effusions were correctly classified against the histopathological diagnoses. At the same cut-off concentration, 9 out of 10 [sensitivity=90% (95% CI: 55.5-98.3%); specificity=57.9% (95% CI: 33.5% -79.7%)] infective effusions were correctly classified against microbiological diagnoses. The positive likelihood ratio (LR+) and negative likelihood ratio (LR−) are 5.79 and 0.47 at this cut-off concentration. For the modified Light's criteria, there is no documented use to further classify the pleural fluid into malignant or infective causes. The positive and negative predictive values for pleural fluid DNA to classify malignant effusions from exudative effusions (including both malignant and infective effusions) are 91.6% and 52.9%, respectively.

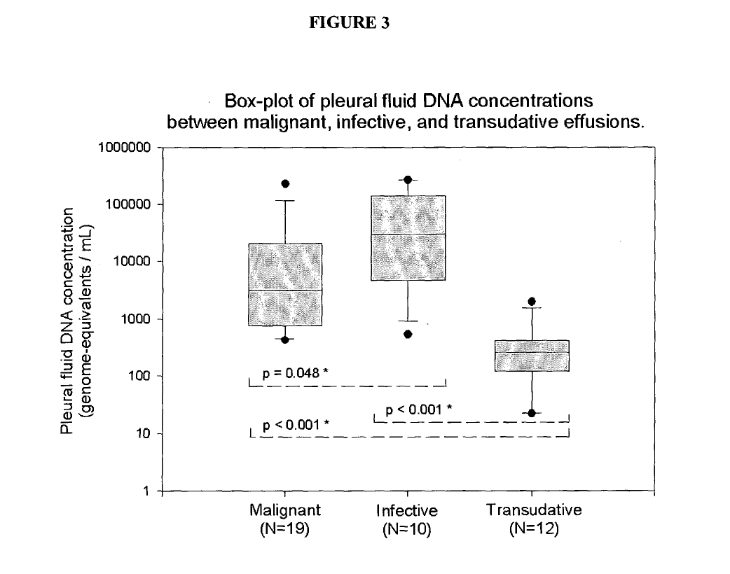

The quantitative results for the pleural fluid DNA concentration between exudative (including both malignant and infective causes) and transudative effusions are illustrated as shown in FIG. 3. There were significant differences in the pleural fluid DNA concentrations between malignant and transudative (p<0.001), malignant and infective (p=0.048) as well as infective and transudative (p<0.001) groups.

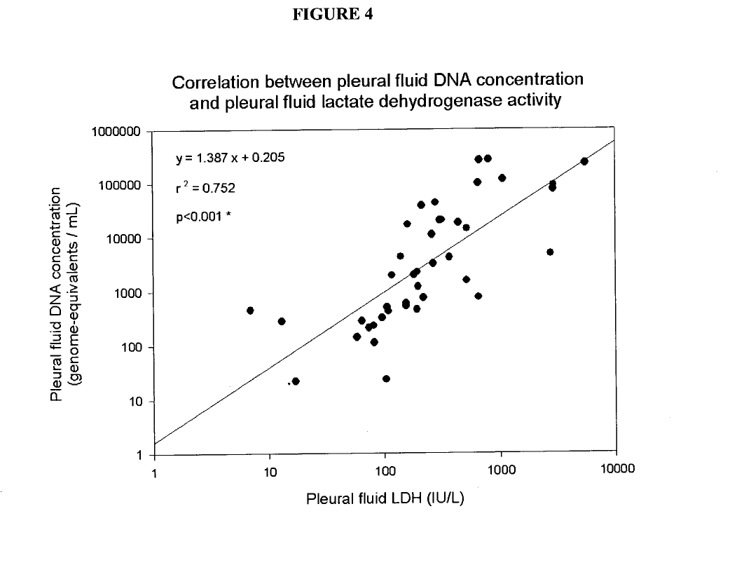

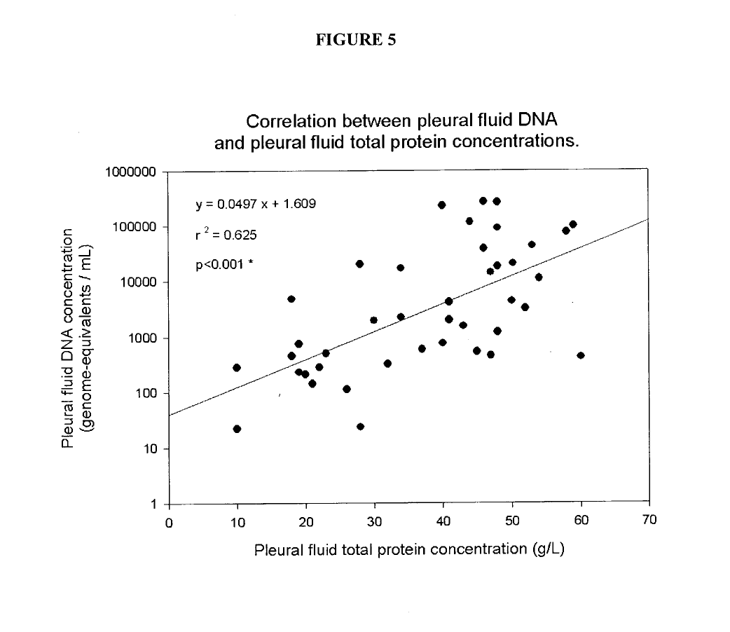

There were significant correlations between pleural fluid DNA concentration and pleural fluid lactate dehydrogenase activity (r²=0.752; p<0.001) as well as pleural fluid DNA and pleural fluid total protein concentrations (r2=0.625; p<0.001) as shown in FIGS. 4 and 5 respectively.

The present invention provides a simple and highly accurate method for testing pleural fluid for nucleic acids for the classification of pleural effusions. Using the methods of the present invention, pleural fluid DNA was detected in varying concentrations in the pleural fluid of subjects suffering from different types of pleural effusions.

### Example 5

Use of Nucleic Acids Other Than the β-globin Gene as Marker for Pleural Fluid DNA

Theoretically, a skilled practitioner can use any genomic sequences to reflect the amount of pleural fluid DNA present in the pleural fluid. To apply other sequences into the analysis of pleural fluid DNA, the skilled practitioner can repeat the DNA extraction steps for pleural fluid using QIAamp Blood Kit (Qiagen, Hilden, Germany) by use of the blood and body fluid protocol according to the manufacturer's recommendations. The volume of pleural fluid used for DNA extraction is 600 to 800 μL per column.

Examples of nucleic acids species that can be used in the methods of the present invention include, but are not limited to, the human leukocyte antigen (HLA) locus (for example, nucleotide sequences found in the following GenBank accession numbers: AF541998, AF539618, AJ507393, AJ507391, AJ507394, AJ507390), Y chromosomal genes (for example, nucleotide sequences found in the following GenBank accession numbers: BC034942, NM_{—}002791, AF517635, NM_{—}004676, NM_{—}004081, NM_{—}139214) and (Lee T H et al., Transfusion 2001;41:276-282), blood group antigen genes like RHD (for example, nucleotide sequences found in the following GenBank accession numbers: NM_{—}016225, NM_{—}016124, NM_{—}138617, Z97026, NM_{—}138618, NM 020485) and (Lo Y M D et al., N. Engl. J. Med. 1998;339:1734-1738), and mitochondrial DNA (for example, nucleotide sequences found in the following GenBank accession numbers: NM_{—}005002, NM_{—}004550, NM_{—}003645, NM_{—}002491, NM_{—}005917, NM_{—}005984) and (Zhong S et al., J. Clin. Pathol. 2000;53:466-469) and mRNA (Poon L L M et al., Clin. Chem. 2000;46:1832-1834; Chen X Q et al., Clin. Cancer Res. 6:3823-3826). Probes and primers for the detection of other known nucleic species in the pleural fluid of a subject suffering from a pleural effusion can be synthesized using well known techniques.

Real-time PCR amplification can be performed and the resultant data are compared to a control. The volume of extracted pleural fluid DNA used for amplification is 5 μL. Real-time quantitative PCR is performed by use of an Applied Biosystems 7700 Sequence Detector (Applied Biosystems, Foster City, Calif., USA) or any state of the art analyzer from various manufacturers, but not limited to Applied Biosystems 7700 Sequence Detector. Duplicate analyses are performed for each sample, and the mean result is used for further analysis. A calibration curve is analyzed in parallel with each assay. Double-distilled water is used as the negative control for quantitative real-time PCR.

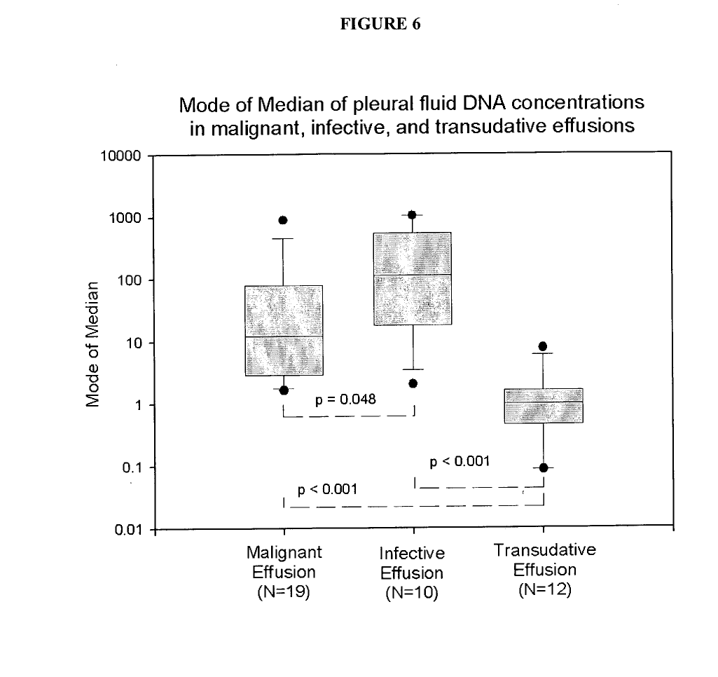

Due to the variability in instrument sensitivity, slope of calibration curve, and reagent reactivity, the cut-off concentration may vary from laboratory to laboratory. However, a skilled practitioner can solve this problem by transferring any data for the representative DNA marker sequence concentration into multiples of median (MoM), wherein the median refers to the median of any DNA marker sequence concentrations from a respectable number of known transudative effusions that can be determined at the same experiment or previously in a separate experiment. Using the data of the β-globin gene as an example, a table of MoM and a box-plot of MoM against different classes of pleural effusions are constructed in Table 2 and shown in FIG. 6 respectively.

TABLE 2

| Malignant effusions | Infection effusions | Transudative effusions |
|---|---|---|
| Pleural fluid DNA | Pleural fluid DNA | Pleural fluid DNA |
| conc. (MoM) | conc. (MoM) | conc. (MoM) |
| 1208.25 (4.662) | 535.5 (2.07) | 143.5 (0.55) |
| 749.75 (2.89) | 38523.9 (148.63) | 22.375 (0.09) |
| 76264.63 (294.24) | 99591.9 (384.24) | 23.75 (0.09) |
| 765.38 (2.95) | 18301.9 (70.61) | 284.38 (1.10) |
| 595.38 (2.30) | 269111 (1038.28) | 456.75 (1.76) |
| 2255.5 (8.70) | 42880.3 (165.44) | 234 (0.90) |
| 20423.25 (78.80) | 4344.5 (16.76) | 112.75 (0.44) |
| 455.75 (1.76) | 4835.13 (18.65) | 2017.25 (7.78) |
| 17210.5 (66.40) | 260831 (1006.33) | 327.5 (1.26) |
| 1982.5 (7.65) | 20641.5 (79.64) | 508.5 (1.96) |
| 1559.38 (6.02) | | 214.25 (0.83) |
| 429.5 (1.66) | | 286.88 (1.11) |
| 4221 (16.29) | | |
| 230747.37 (890.26) | | |
| 14263.5 (55.03) | | |
| 89881 (346.78) | | |
| 11077.13 (42.74) | | |
| 116725.12 (450.35) | | |
| 3192.5 (12.32) | | |

The median of pleural fluid DNA concentration for all transudative effusions is 259.19 genome-equivalents/mL

Data analysis for Spearman correlation, linear regression, and non-parametric Kruskal-Wallis test statistics were performed by the use of SPSS 10.0 for Windows (SPSS). Receiver-Operator Characteristic (ROC) curve was plotted using MedCalc 6.16 statistics program (MedCalc) to determine the best cut-off MoM for pleural fluid DNA. The beauty of using the MoM is the transferability of data across different laboratories as exemplified in the example of Down Syndrome Screening (Haddow J E et al., N. Engl. J. Med. 1998;338:955-961 and Parvin C A et al., Clin. Chem. 1991;37:637-642).

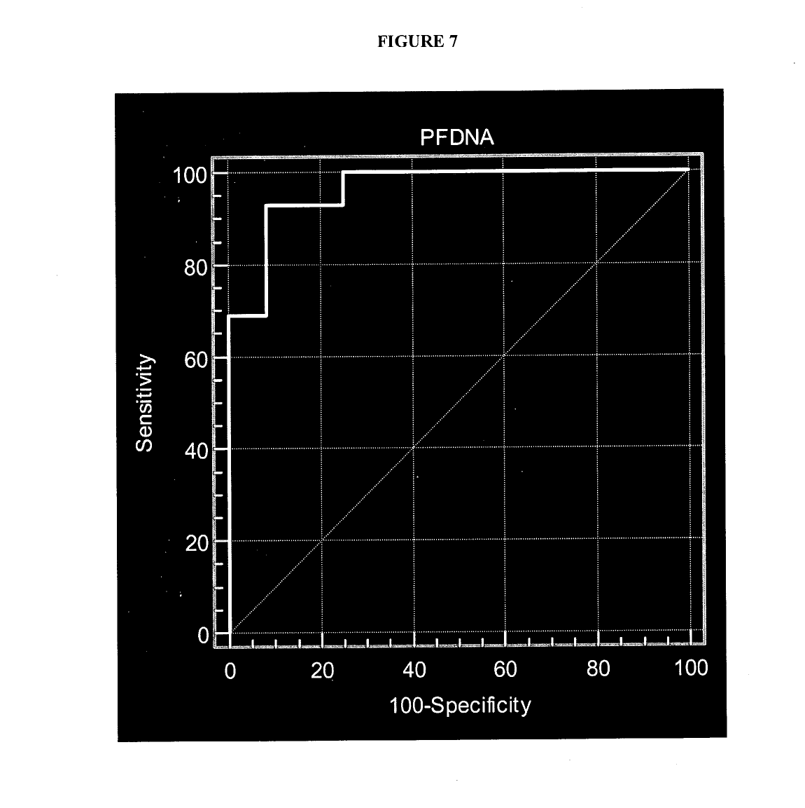

The ROC curve for MoM of pleural fluid DNA concentration was plotted as shown in FIG. 7. The area under the curve is 0.963 [95% Confidence Interval (95% CI): 0.851-0.995]. The best cut-off MoM for pleural fluid DNA concentration was chosen to be 1.96. Pleural fluids with MoM of their respective DNA concentrations equal to or above 1.96 are regarded as exudative effusions while pleural fluids with MoM of their respective DNA concentrations below this cut-off are regarded as transudative effusions.

Using this cut-off MoM, 38 out of 41 [sensitivity=93.1% (95% CI: 77.2% -99.0%); specificity=91.7% (95% CI: 61.5%-98.6%)] pleural effusions were correctly classified into exudative and transudative groups when compared to the gold standard. The positive likelihood ratio (LR+) and negative likelihood ratio are 11.17 and 0.08 at this cut-off MoM. Using the modified Light's criteria, 36 out of 41 [sensitivity=96.6%; specificity=66.6%] pleural effusions were correctly classified into exudative and transudative groups when compared to the gold standard. The positive predictive values for MoM of pleural fluid DNA concentration and modified Light's criteria are 96.4% and 87.5%, respectively. The negative predictive values for MoM of pleural fluid DNA concentration and modified Light's criteria are 84.6% and 88.8%, respectively.

Similarly, MoM cut-off can be determined for the differentiation between malignant effusions and infective effusions from a cohort of patients with pleural effusions as exemplified in paragraphs [0069] to [0070] above.

The quantitative results for MoM of pleural fluid DNA concentration between exudative (including both malignant and infective causes) and transudative effusions are illustrated in FIG. 7. There were significant differences in the MoM of pleural fluid DNA concentrations between malignant and transudative (p<0.001), malignant and infective (p=0.048) as well as infective and transudative (p<0.001) groups.

The result of using MoM is exactly the same as using pleural fluid DNA concentration as cut-off.

It is understood that the examples and embodiments described herein are for illustrative purposes only and that various modifications or changes in light thereof will be suggested to persons skilled in the art and are to be included within the spirit and purview of this application and scope of the appended claims. All publications, patents and patent applications cited herein are hereby incorporated by reference in their entirety for all purposes.

What is claimed is:

1. A method of classifying a pleural effusion in a subject as transudative or exudative, the method comprising:
(i) obtaining a sample of pleural fluids from a patient suffering from a pleural effusion, and
(ii) detecting the concentration of human nucleic acid in the sample, with the proviso that the nucleic acid is not overexpressed in cancer cells and is not telomerase or adenosine deaminase nucleic acid, and
(iii) classifying the pleural effusion as transudative or exudative by comparing the concentration of nucleic acid in the sample to a standard.

2. The method of claim 1, wherein the exudative effusion is further classified as a malignant effusion or an infective effusion.

3. The method of claim 1, wherein the patient is suffering from a disease selected from the group consisting of congestive heart failure, end-stage renal failure, pulmonary tuberculosis, empyema, chest infection, malignant neoplasm, pulmonary embolism, pneumonia, liver disease, kidney disease, and lymphangitis carcinomatosis.

4. The method of claim 1, wherein the nucleic acid in the sample is DNA.

5. The method of claim 1, wherein the nucleic acid in the sample is RNA.

6. The method of claim 4, wherein the DNA is the β-globin gene DNA.

7. The method of claim 1, further comprising the step of amplifying the nucleic acid.

8. The method of claim 7, wherein the nucleic acid is DNA and the DNA is amplified using PCR.

9. The method of claim 8, wherein the DNA is amplified using real-time PCR.

10. The method of claim 7, wherein the nucleic acid is RNA and the RNA is amplified using reverse transcriptase PCR.

11. The method of claim 10, wherein the RNA is amplified using reverse transcriptase real-time PCR.
